(19) United States
(12) Patent Application Publication
(10) Pub. No.: US 2026/0115037 A1
(43) Pub. Date: Apr. 30, 2026

(54) Hannah Cervical Cup

(71) Applicant: Ashley Camille CRAFTON, Nampa, ID (US)

(72) Inventor: Ashley Camille CRAFTON, Nampa, ID (US)

(21) Appl. No.: 19/482,827

(22) PCT Filed: May 6, 2024
(86) PCT No.: PCT/US2024/027949

§ 371 Date: Nov. 10, 2025

Related U.S. Application Data
(60) Provisional application No. 63/465,587, filed on May 11, 2023.

(51) Int. Cl. A61F 6/08

(52) U.S. Cl. CPC A61F 6/08

(57) ABSTRACT

A cervical cup includes a flexible body having a flange to treat preterm birth, the cervical cup being configured to apply suction pressure to the cervix of a patient by compressing the flexible body and holding the flange against the pelvic floor of a patient being treated for preterm birth, the suction pressure being generated by the release of the compression applied to the flexible body. An example of the disclosed methodology to treat preterm birth includes inserting a cervix into a cervical cup and applying a vacuum thereto so as to secure the cervical cup to the cervix.

### CROSS-REFERENCE TO RELATED APPLICATIONS

This application claims the benefit of International Application No. PCT/US2024/027949, filed May 6, 2024, which claims priority to U.S. Patent Application Ser. No. 63/465,587, filed on May 11, 2023, entitled “Hannah Cervical Cup,” the contents of which are herein incorporated by reference in their entirety.

### BACKGROUND

### Technical Field

Embodiments of the subject matter disclosed herein relate generally to apparatuses, methods and systems and, more particularly, to devices, processes, mechanisms and techniques for the treatment of preterm birth.

### Description of Related Art

Every year, an estimated fifteen million babies are born preterm (before 37 completed weeks of gestation), and this number is rising. Preterm birth complications are the leading cause of death among children under five years of age and responsible for approximately one million deaths in 2015. Those who survive often suffer from debilitating disabilities such as developmental, learning, visual, and hearing impairments. Across the world, the rate of preterm birth ranges from 5% to 18% of all births. Experts have declared that preterm birth is, in quantity and severity, the most important contributor to perinatal morbidity and mortality both in well- and low-resource countries and, of course, is impacting the health of families around the globe. Preterm birth happens for many known and unknown reasons.

The cervix plays an important role in pregnancy and birth. One complication that leads to preterm birth is cervical insufficiency (a medical condition of pregnancy in which the cervix begins to dilate (open) and efface (thin) in the absence of contractions; thus, the cervix cannot retain the pregnancy to full-term). The main function of the cervix is to keep the growing fetus within the uterus for the extent of pregnancy and then to facilitate the removal of the fetus during delivery. This requires the cervix to withstand multiple forces from the uterus, growing fetus, and amniotic sac. Then, in a complete role reversal, the cervix softens, shortens, and dilates to allow for the delivery of the uterine contents. The functionality of the cervix is primarily derived from the collagen that makes up the bulk of its body. Collagen is a ubiquitous protein responsible for maintaining the structural integrity and mechanical function of tissues in most multicellular organisms. It has a fibrillar structure that is able to resist tensile, shear, or compressive forces and is found in structural tissues such as skin, tendons, ligaments, blood vessels, cartilage, bones, teeth, and the cervix. The collagen of the cervix is directionally layered to compose an epithelial and a stromal region. Fibroblasts, smooth muscle cells, immune cells, endothelial cells, and nerve fibers also reside in the stromal region. The stromal fibroblasts synthesize the collagen-rich extracellular matrix, and thus the stroma is the load-bearing connective tissue layer, making up approximately 53-70% of the tissue.

There are at least three main conventional methods of treatment for spontaneous preterm birth prevention: cerclage, progesterone supplementation, and pessary application.

Cervical cerclage refers to a variety of procedures that use sutures or synthetic tape to reinforce the cervix during pregnancy with women who have a history of a short cervix or cervical insufficiency in a prior pregnancy or with women who present with a short cervix (a “rescue” cerclage). There are several different techniques for cerclage placement, but the general idea is that a non-absorbable suture is placed during surgery to tie closed the cervix. Cervical cerclage can be done through the vagina (transvaginal cervical cerclage) or, less commonly, through the abdomen (transabdominal cervical cerclage). They can be placed pre-conceptually, after conception, or as a “rescue” cerclage (after pregnancy has begun and the discovery of an insufficiency has been made). The transabdominal approach increases the neonatal survival rate from 43% to 71% and typically increases the gestational age at delivery from 25 to 30 weeks. Unfortunately, cerclages have many drawbacks. Transvaginal cerclages have the drawback of not pulling uniformly on the cervix and having the risk of “tearing through” the cervix, which can undermine the integrity of the already weakened cervix. Transabdominal cerclages have proven to be more effective, but, given their more invasive nature, are almost impossible to place after pregnancy has been established; they cannot be used as a type of rescue cerclage and must be placed only for patients who have a historical diagnosis. Cerclages have proven to be ineffective in multiple gestational pregnancies, are invasive, and must be placed under the presence of anesthesia.

Progesterone supplementation, which can be performed via weekly intramuscular injections or per regular vaginal suppository, is by itself controversial and its efficacy has been debated. A recent study found that this treatment is not beneficial, suggesting that the role of progesterone may be more limited than previously thought.

A pessary is an apparatus (a ring-like device) that is inserted into the vagina to reduce the protrusion of pelvic structures into the vagina by the application of a localized positive pressure on the cervix. Pessaries are of varying shapes and sizes. One traditional pessary type is the Arabin pessary, which was first produced and used in the 1970s in Germany. Several other types of pessary have been used in an attempt to prevent preterm birth, but none have been very successful. The Arabin pessary also has mixed literature regarding its efficacy use; therefore, it is not a common treatment for cervical insufficiency in the United States. Most practitioners use a combination of therapies to treat cervical insufficiency. Even a combination of therapies has proven controversial, as no randomized trials have evaluated the efficacy of a combination therapy.

Another complication that leads to preterm birth is Preterm Pre-labor Rupture Of Membranes (PPROM). PPROM is the spontaneous or iatrogenic rupture of the fetal amnion (“water breaking”) prior to the fetus reaching full gestation that is unaccompanied by contractions. PPROM is responsible for, or associated with, approximately one-third of preterm births and the single most common identifiable factor associated with preterm delivery. The treatment for PPROM is less straightforward than its diagnosis and is a source of controversy throughout perinatal medicine. The treatment of PPROM depends on many factors, including whether or not the patient is (1) showing signs of infection (fever, increase in leukocytes, septicemia, purulent vaginal drainage, etc.); (2) at risk for a prolapsed umbilical cord and/or placental abruption; or (3) mother or fetus are showing signs of distress. However, PPROM is strongly associated with oligohydramnios (little amniotic fluid) and anhydramnios (no amniotic fluid) and early, sever, prolonged oligohydramnios (and anhydramnios) can be associated with pulmonary hypoplasia, facial deformation, and orthopedic abnormalities. Each day and week that passes, the risks and benefits of prolonging the pregnancy must be weighed and the plan of care adapted accordingly.

Expectant management is the most commonly used treatment for PPROM. It usually includes at least some initial hospitalization and often hospitalization until delivery to effectively manage the timely assessments and care of mother and fetus, including, but not limited to, frequent vital sign assessments and fetal heart rate assessments. Hospitalization can also help limit the outside activities of the mother (such as work, family, and household duties), thereby mitigating risks associated with such tasks. Most practitioners use a combination of such treatments to address the diagnosis of PPROM.

Another complication that leads to preterm birth is preterm labor. Preterm labor is the presence of uterine contractions that lead to cervical change prior to 37 completed weeks of gestation. Preterm labor is another difficult phenomenon to diagnose, and its cause is unknown, but it is probably multifactorial and different for every woman. Some women with this diagnosis deliver preterm and some are able to carry the pregnancy to term. Data on the number of women who are diagnosed with preterm labor and continue the pregnancy until term are unclear. Usual treatment includes all treatments mentioned previously for both cervical insufficiency and PPROM and can include progesterone supplementation, pessary use, corticosteroids, tocolytics, intravenous hydration, treatment of underlying infections, bed rest, and hospitalization. Treatment for these patients proves difficult because of the aforementioned problems but also because many tocolytics are not proven to be effective or beneficial to both maternal and neonatal outcomes.

However, despite the above-summarized progress in treating preterm birth to date no devices, processes, and/or methods exist that are capable of treating these conditions without (1) undermining the integrity of an already weakened cervix, (2) being so invasive in nature, (3) being more effective in multiple gestational pregnancies, (4) unnecessarily exposing mother and baby to prolonged use of controversial (often times off-label) drugs of at least questionable efficacy, (5) reducing the need of hospitalization during treatment, or (6) reducing the risk of infections, while allowing for prolonged use of the treatment, minimizing the required hospitalization, and/or strengthening a woman's cervix at the same time that preterm birth is being treated.

### SUMMARY

Apparatuses, methods, and processes for treating preterm births address one or more of the above-summarized needs or others known in the art. As disclosed herein, such apparatuses include a cervical cup having a body, a flange attached to the body at one of its end portions, and a vacuum port disposed at the other end portion of the body so that application of a vacuum to the vacuum port will secure the cervical cup to a cervix disposed inside of the body.

Methods for treating preterm births include inserting end portions of a cervix into a body of a cervical cup having a flange and a vacuum port and applying a vacuum to the vacuum port so as to secure the cervical cup to the cervix disposed inside of the body.

### BRIEF DESCRIPTION OF THE DRAWINGS

The accompanying drawings (not drawn to scale), which are incorporated in and constitute a part of the specification, illustrate one or more embodiments and, together with the description, explain these embodiments. In the drawings:

FIG. 1 illustrates an exemplary embodiment of a cervical cup according to one aspect of the subject matter disclosed;

FIG. 2 illustrated the selection of the shape of a cervical cup according to another aspect of the subject matter disclosed taking into consideration the shape of a cervix of a patient to be treated;

FIG. 3 illustrates another exemplary embodiment of a foldable cervical cup according to another aspect of the subject matter disclosed;

FIG. 4 illustrates examples of foldable cervical cups according to another aspect of the subject matter disclosed;

FIG. 5 illustrates another exemplary embodiment of a cervical cup with a plurality of suction holes according to another aspect of the subject matter disclosed;

FIG. 6 illustrates exemplary embodiments of a cervical cup with the localized placement of a plurality of suction holes to better shape a cervix of a patient according to another aspect of the subject matter disclosed;

FIG. 7 illustrates an exemplary embodiment of a pressure control apparatus according to another aspect of the subject matter disclosed;

FIG. 8 illustrates typical pressure-versus-time profiles applied to a patient's cervix using the pressure control apparatus of FIG. 7 according to another aspect of the subject matter disclosed;

FIG. 9 illustrates exemplary embodiments of cervical cups with streamlined tips configured to facilitate insertion in a patient according to another aspect of the subject matter disclosed;

FIG. 10 illustrates another exemplary embodiment of a streamlined embodiment with space provided to allow a vacuum line to be inserted and rotated to connect with a luer-lock type of connection according to another aspect of the subject matter disclosed;

FIG. 11 illustrates another exemplary embodiment of an inflatable cervical cup according to another aspect of the subject matter disclosed;

FIG. 12 illustrates an exemplary embodiment of an applicator for a foldable cervical cup according to another aspect of the subject matter disclosed;

FIG. 13 illustrates exemplary embodiments of straight and curved applicators for a foldable cervical cup according to another aspect of the subject matter disclosed;

FIG. 14 illustrates an exemplary embodiment of a foldable cervical cup inserted into one of the applicators of FIG. 12 according to another aspect of the subject matter disclosed;

FIG. 15 illustrates an exemplary embodiment of a tool for the removal of a cervical cup according to various other aspects of the subject matter disclosed;

FIG. 16 illustrates another exemplary embodiment of a tool for the removal of a cervical cup according to various other aspects of the subject matter disclosed; and

FIG. 17 illustrates a flowchart of a method to treat preterm births according to an embodiment of the subject matter disclosed.

### DETAILED DESCRIPTION

The following description of the exemplary embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to the terminology and structure of apparatuses, systems, or methods for treating preterm births. Nevertheless, those of ordinary skill in the applicable arts will appreciate that the subject matter disclosed herein has other beneficial applications, such as, but not limited to, other tissues in need of strengthening, stiffening, or wrinkle mitigation (such as lips, neck, skin, organ regrowth, wound closure, improvement of keloids, improvement of scar tissue, and others).

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification does not necessarily refer to the same embodiment. Further, the particular features, structures, or characteristics may be combined in any suitable manner in one or more embodiments. Several of the features disclosed in U.S. Pat. No. 11,311,409 can be advantageously combined with the subject matter disclosed here; therefore, the contents of that patent are herein incorporated by reference in their entirety.

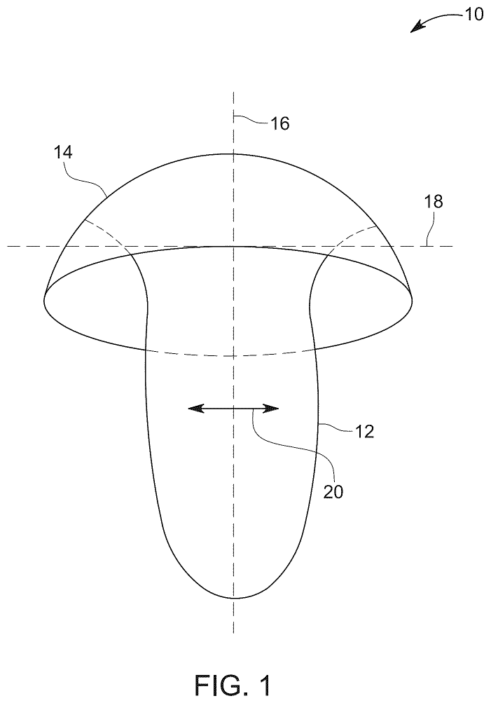

FIG. 1 illustrates an exemplary embodiment of a cervical cup system 10 according to one aspect of the subject matter disclosed. In this illustrated embodiment, the cervical cup system 10 comprises a cervical cup 12 having an inverted flange 14 designed to create support for a pelvic floor of a patient. The cervical cup 12 is shaped in the general form of a cervix, similar to a cervical cap, a menstruation cup, or a diaphragm. The main body of the cervical cup 12 generally extends along an axial axis 16 with the inverted flange 14 generally disposed around a radial axis 18, as shown in FIG. 1. The cervical cup 12 is made of a flexible material such that before being applied the cup is compressed in the general direction of arrow 20 in FIG. 1 and inserted into the vaginal canal with the flange 14 held against the pelvic floor of the patient while releasing the compression applied to the cup. As the cup expands back to its original shape, a self-generated vacuum is created, and the cup is held in place with the generated suction against the pelvic floor, thereby applying a gentle negative pressure or vacuum to the cervix of the patient.

The cervical cup 12, when applied, helps the cervix to remain closed, thick, and high, thus allowing the pregnancy to be extended in the face of the above-noted challenges, including cervical insufficiency, PPROM, and preterm labor. One of the advantageous benefits of the cervical cup 12 is that its use will prolong pregnancy. The prolongation of pregnancy for many women will mean a reduction in deaths, problems, and healthcare costs associated with neonatal and long-term healthcare.

Those of ordinary skill will also appreciate that the size and shape of the cervical cup 12 may vary and will depend on many factors such as whether or not the woman is pregnant for the first time, how far along in the pregnancy, the current health of the cervix, any history of cervical procedures (such as Loop Electrical Excision Procedure (LEEP), cryotherapy, or cone biopsy), the woman's own anatomical variances, and other factors. Typically, an inner diameter of the cervix cup 12 can vary approximately from 4 to 6 cm and the length of the cup may be approximately 7 cm long. However, those of ordinary skill will understand that the physical size and shape of the cervical cups being disclosed herein should in no way limit the subject matter claimed and selection of those parameters may very well be determined on a desired outcome and/or the geometry of a patient's cervix.

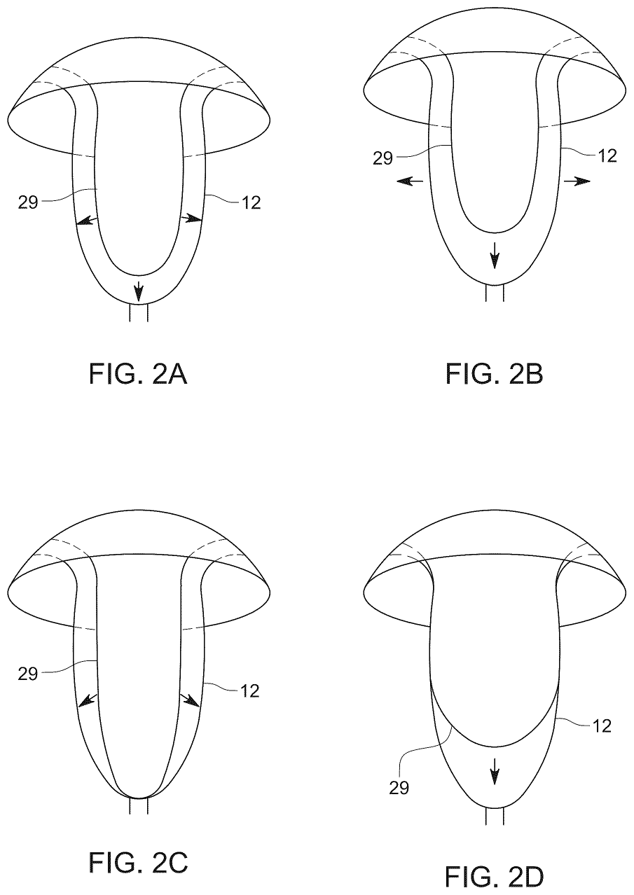

FIG. 2 illustrates qualitatively the size selection of a given cervical cup 12, considering the size of a cervix 29 and the desired cervix-shaping outcome. FIG. 2(A) illustrates a size difference between the cervical cup 12 and the cervix 29 that will allow a nominal pull of the cervix both in the radial and axial directions (as illustrated by the arrows in that figure)—for a given applied vacuum pressure, the amount of pull being determined by the volume between the cervical cup and the cervix before vacuum is applied. If, for a given patient, the cervix is stiffer than normal and/or more suction pull is desired for the shaping of the cervix, the volume between the cervix and cervical cup can be increased by providing a cervical cup that is larger than the cervix as shown in FIG. 2(B). As this happens, the viscoelastic nature of the cervix tissue allows the tissue to be displaced into the shape of the cup. Softer tissue will be displaced into the cup with a lower applied vacuum pressure, and stiffer tissue will require a higher vacuum pressure. FIG. 2(C) illustrates a cervix and cup combination that will produce only a radial pull and FIG. 2(D) a combination that will provide only an axial pull at the end portion of the cervix.

The cervical cup 12 can be placed as early as the 13th week of pregnancy after the risk of miscarriage has abated. It may be placed on the cervix at any time between the 13th and 37th week of pregnancy. The cervical cup 12 is designed to be placed on the cervix to prevent preterm birth as a primary treatment following the history of preterm birth in the mother or as a preventive treatment for potential preterm birth concerns in first-time mothers. It is also designed to be placed on the cervix as a secondary treatment for preterm birth at the time a short cervix is identified, at the time the mother presents with preterm labor symptoms, or at the time the mother presents with rupture of membranes. One of the advantageous features of the cervical cup 12 is that it may stay in place until delivery, ideally around the 37th week of gestation.

The combination of the cervical cup's unique structure and suction enables the cervix—made up of significant amounts of collagen—to enhance its natural load-bearing qualities by placing the collagen under tensile strain. All while keeping the cervix closed and elongated, even during uterine contractions, the distinctive shape of the cup's flange offers pelvic floor support for the growing baby and the womb.

One of the effective ways to improve collagen stiffness is through the application of negative pressure. This negative pressure creates tension within the collagen. Negative pressure on collagen produces stiffness in two ways: immediate stiffness and a long-term improvement in collagen function. The ability to apply negative pressures to the cervix is one of the beneficial features of the subject matter disclosed herein.

Despite all the diverse and complex etiologies of preterm birth, premature cervical remodeling (softening) remains the one common feature of all spontaneous preterm births. Women with a history of premature cervical failure that resulted in spontaneous preterm birth have been shown to have decreased collagen concentration and collagen crosslink maturity ratios within the cervix and models of preterm birth demonstrate a decrease in cervical stiffness. The subject matter disclosed herein focuses on cervical ripening as the most logical place to concentrate in order to solve the worldwide problem of preterm birth.

The softening of the extracellular matrix is the impetus for delivery through the cervix. Studies have demonstrated that the internal cervical os functions as a sphincter, with a ring of cervical smooth muscle cells located at the level of the junction of the lower uterine segment and the internal cervical os. This ring of cervical smooth muscle contracts, keeping the internal os closed for the duration of pregnancy, and then opens when cervical softness increases. This softening is part of a biochemical and biomechanical process that breaks down the collagen structures and crosslinks. This cervical softening allows for the relaxation of the cervical smooth muscle ring leading to cervical effacement and dilation, which culminates in birth—the expulsion of uterine contents.

When the stiffness of the extracellular matrix increases, the contractility of the internal os sphincter increases (meaning that it stays more tightly closed). Sphincter malfunction due to a weak extracellular matrix can be prevented with an improvement in the stiffness of the extracellular matrix, which will, in turn, decrease preterm birth.

As the triple helix of mature collagen fibers unfurls under tension, a change in state occurs, creating immediate tissue stiffness. Research has shown that the cervical tissue response under tension is much greater than the tissue response under compression—the tissue becoming about an order of magnitude stiffer under tension compared to the softening that occurs when the cervix undergoes compression. This asymmetry is partly due to the collagen fiber network, where collagen fibers support loads under a tensile strain. The nonlinearity in the tension/compression stress response is typical of soft hydrated tissues within a collagen fiber network, where collagen fibers resist tension and preferentially align to create deformation resistance. In the case of the cervix, collagen fibers under tension have a preferred circumference orientation to best bear the load. Deformation of tissue under tension elicits biochemical and motility responses as well in order to best sustain the tension.

Over time, there is an improvement in collagen function under tension. Research surrounding all types of collagens has shown that moderate amounts of long-term mechanical loading increase collagen tensile strength, increase collagen synthesis, decrease collagen degradation, decrease adhesions, and has even been shown to decrease inflammatory mediators such as prostaglandins—important hormone-like proteins that mediate the timing of birth.

This long-term tension causes collagen fibrils to reorient towards the direction of the loading permanently. Mechanical strengthening from loading induces stabilizing changes internal to the fibrils themselves and in fibril-fibril interactions. These mechanisms strengthen the mechanical ability of the tissue to carry a load. This growth (change in mass) and remodeling (change in microstructure) that occur together during long-term exposure to tension are driven by the tissue seeking a preferred homeostatic state. A biological growth and remodeling process addresses long-term tension to restore normalcy. This tension also causes a change in the production of biomolecules and their receptors that support the process of growth and remodeling. Loading of collagen also creates length, even when tissue is at rest due to improvements in tissue geometry. A long cervix is associated with term births. Further, tissue integrity improves so that when more strain is applied, collagen fibrils are strain stabilized against matrix metalloproteinase (tissue degrading enzyme) degradation and the collagen experiences a less stressful state. Stretch has been shown to up-regulate collagen and other extracellular matrix protein production in a vascular smooth muscle, leading to significantly greater collagen levels. These long-term, incremental changes are then preserved with increased cross-linking stimulated by the stretch. According to the subject matter disclosed herein, cervical cups are designed to provide these benefits to a weak cervix.

The disclosed cervical cups also have the ability to maintain a pregnancy following early rupture of the fetal membranes by providing a physical barrier to infection—a leading cause of preterm birth—and allowing amniotic fluid to reaccumulate—an important factor in the health of an unborn baby.

Those of ordinary skill in the applicable arts will appreciate that the disclosed devices have the ability to prolong pregnancy for weeks. These cervical cups may be applied after the 13th week of pregnancy by a physician in an outpatient or inpatient setting as needed and is designed to be removed when the pregnancy reaches term (37 weeks or more of pregnancy). Only a few minutes of time is required for both placement and removal. The benefits of the disclosed cervical cups include placing the cervical tissue itself in a state of ripening-resistant tension; creating pelvic floor support; creating a barrier to infection; creating an environment where amniotic fluid can reaccumulate; offering a mode of medication administration that is conducive to maintaining the pregnancy; and/or maintaining all of the noted benefits, and others, for the length of gestation.

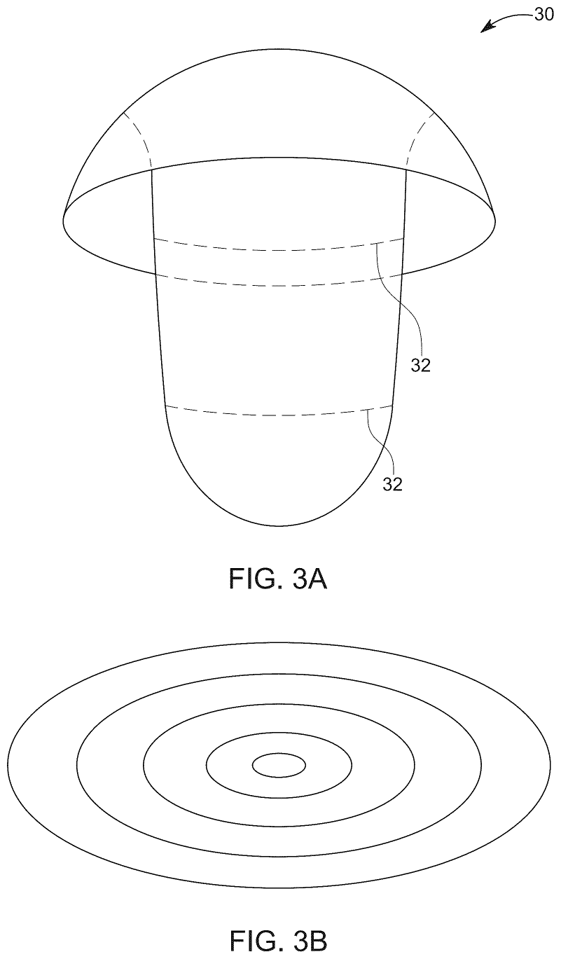

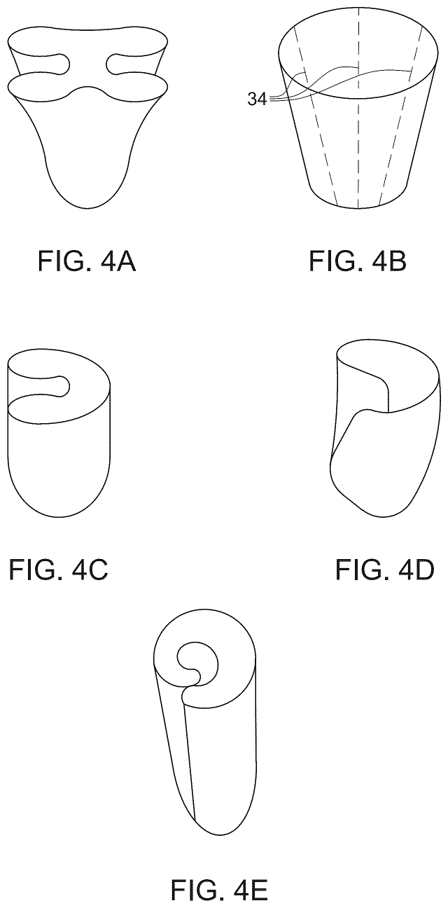

FIG. 3 illustrates an embodiment of a foldable cervical cup system 30 with the cup extended in FIG. 3(A), and the cup collapsed as shown in FIG. 3(B). In this foldable embodiment, one or more radial fold lines 32 are provided on the body of the cervical cup in order to allow the cervical cup to collapse along the fold line 32, as shown in FIG. 3(B). FIG. 4 illustrates various embodiments of how the body of a cervical cup can be made to fold in order to facilitate insertion during treatment. In the embodiment shown in FIG. 4(A) and 4(B), axial fold lines 34 are provided in order to facilitate folding the body of the cup axially. In FIG. 4(C)-4(E), the body of the cups is made to wrap around itself so as to reduce the size of the cup before insertion through the vagina.

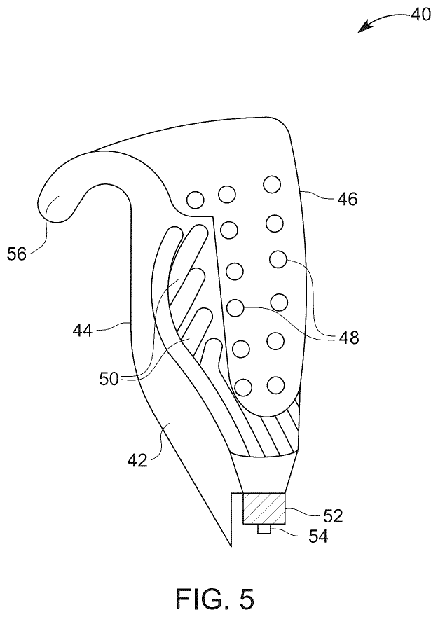

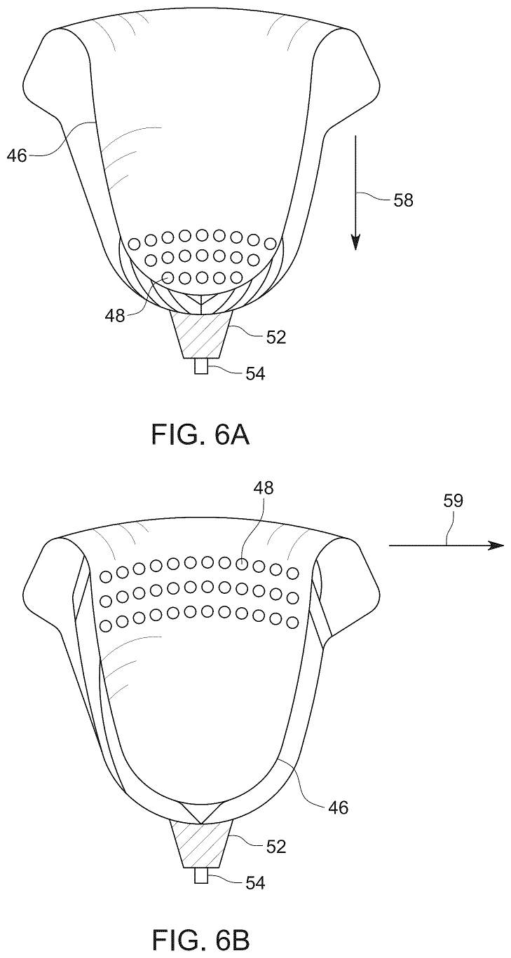

In order to facilitate the control of the pressure applied to the cervix, another embodiment of a cervical cup system 40 is illustrated in FIG. 5. In this illustrated embodiment, the cervical cup system 40 comprises a cervical cup 42 having an outer wall 44 and an inner wall 46; this inner wall 46 having a plurality of suction holes 48. Vacuum is applied to the plurality of suction holes 48 via suction lines 50 connected to a valve 52 disposed at the distal end portion of the cervical cup system 40. A vacuum source (not illustrated) is connected to valve 52 via a vacuum hose 54. As also shown, the cervical cup system 40 further comprises a flange 56 attached to a proximal end portion of the cervical cup 42, the flange 56 being solid in some embodiments and hollow in others. Application of a vacuum to the valve 52 will cause the cervix to be displaced toward the various suction holes 48, thereby holding the cervix to the cervical cup. Placement of the suction holes 48 in different parts of the inner wall 46 will allow for better control of the pull of the cervix, as shown in the two exemplary embodiments in FIG. 6(A) and 6(B). Placing the suction holes toward the distal portion of the inner wall 46 will facilitate pulling the cervix tissue down (as illustrated by arrow 58 in FIG. 6A) to better shape the cervix. Placement of the suction holes 48 toward the proximal portion of the inner wall 46 will facilitate pulling the endocervix out, as shown by arrow 59 in FIG. 6B. Therefore, different distributions of the suction holes 48 will allow the cervix tissue to be shaped as desired by the location of the suction holes 48. Although not illustrated in FIGS. 5 and 6, those of ordinary skill in the application arts will appreciate that embodiments having different suction lines 50 connected to different vacuum sources are possible and may be desirable for better control of the pressure applied to the cervix as further explained below.

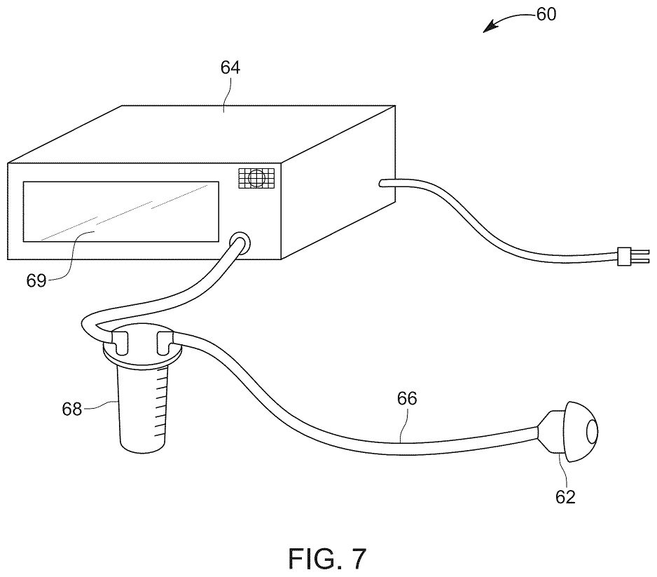

With a pressure control apparatus 60, shown in FIG. 7, it is possible to automatically adjust the negative pressure amount applied to different cervixes depending on their viscoelastic properties. For example, more pressure can be applied to a cervix with stiffer tissue while less pressure can be applied to a cervix with delicate tissue of a softer constitution.

As shown in FIG. 7, a cervical cup 62 according to an embodiment of the subject matter disclosed is attached to a pumping control system 64 via a tube 66 connected to a filter or suction canister 68 so as to prevent backfill of substances from the cervix attached to the cervical cup 62 into the pumping control system 64. The vacuum may be turned on once the cervical cup 64 is placed on the tissue. The pumping control system 64 is configured to apply a vacuum pull to the cervical cup 62 in a controlled fashion, as will be further explained below. As this happens, the viscoelastic nature of the tissue allows the tissue to be shaped into the geometry of the cervical cup 62. Softer tissue will be displaced into the cup with less volumetric pull from the pumping control system 64, and stiffer tissue will require a larger amount of volumetric pull. Once the tissue reaches the inlet of the vacuum (either via single or multiple suction holes 48), the cervical tissue seals off the inlet to the vacuum tubing. This creates a pressure change within the tubing. A manometer (electronic or otherwise not illustrated in FIG. 7) in the pumping control system 64 detects a drop in pressure in the tubing connected to the valve 52 of the cervical cup 62 once the cervical tissue blocks the suction hole(s) 48. This can be detected by a drop in the vacuum tubing pressure alone or by comparing that pressure to a reference pressure value (e.g., the pressure of an ambient or atmospheric channel port set to ambient open air within the pump box itself, along the pump tubing, or in another location near the cervical cup if a double vacuum hose 54 is used. Therefore, in some embodiments, a change in the aspiration rate caused by the closure of the pump tubing is detected, while in other embodiments a comparison between two different pressures is used for that determination. Once this point is detected, an audio and/or visual signal will be triggered in the pumping control system 64 to inform the user, the pump will then switch to a mode that holds at the desired pull achieved, and the amount of volumetric pull or pressure will be recorded and/or displayed at a display 69 of the pumping control system 64. This process will signal the user to disconnect the vacuum tubing from the base of the cup at the check valve and will allow the cup to stay on the tissue with an amount of negative pressure appropriate for the level of stiffness or softness of the particular cervix tissue.

As previously noted, the tube 66 can be (a) a single tube connected to the valve 52 and suction lines 50 of the cervical cup or (b) a double tube, each portion connected to different suction lines 50 of the cervical cup or one portion connected to a suction line or lines in the cup and the other connected to a reference ambient pressure. A double-walled tube will facilitate the detection of a change in pressure when the cervix tissue reaches a desired area of the inner wall of the cervical cup. In some embodiments, the tube 66 may be further comprised of a guide wire in order to better place and remove the cervical cup. In other advantageous embodiments, the cervical cup is provided with only two suction holes 48, each connected with one line of a double suction line 50. Each suction hole 48 is disposed in selected locations to optimize the determination of the cervix's position inside the cervical cup and the application of the desired pressure level to the cervix.

Those of ordinary skill in the applicable arts will appreciate that it takes a stronger force to move a stiffer tissue and a weaker force to move a softer tissue. In use, the cervical cup 62 of the pressure control apparatus 60 is placed on the viscoelastic tissue of a patient's cervix and suction is initiated and the cervix tissue begins to be displaced into the cervical cup 62. When the tissue is displaced to the point that it fills the cup, the tissue occludes the check valve, creating a change in pressure in the vacuum tubing, which is detected by the volumetric pull device and induces it to hold steady at that particular volume until the check valve is disconnected from the tubing and the cup is left in place. In other words, the vacuum is deployed until the tissue is displaced enough to fill the cup. If the cup is fitted to each specific tissue shape and only displaces the tissue by a set volume (being the space between the cup wall and the tissue's original shape), stiffer tissue is placed under a greater amount of negative pressure, while softer tissue is placed under a lesser amount of negative pressure, because stiffer tissue requires more force to displace.

As previously disclosed, If there is more space to fill between the cup wall and the original shape of the tissue, this will induce a greater amount of negative pressure on the tissue than one where the cup wall is in closer proximity to the tissue. This makes it possible to control the amount of negative pressure on the tissue by changing the volumetric amount the tissue is displaced. More space to fill will result in a larger force while less space will result in less force applied to the cervical tissue. In other words, depending on the softness or stiffness of the patient's cervix tissue and/or the size of the cervical cup used, a different level of vacuum will be required to obtain the desired effect of shaping the patient's cervix. These two factors make it possible to control the amount of pressure exerted on the tissue by adjusting the amount of space between the original tissue shape and the cup wall while still exerting more force on stiff tissue and less on soft tissue.

A combination of the correct cervical cup shape and the pressure control system 60 can be used to apply the desired amount of vacuum to shape the cervix for a particular patient. Those of ordinary skill will appreciate that one can deform or displace soft tissue with an amount of negative pressure specifically tailored to the stiffness or softness level of the tissue itself and measure the level of stiffness of the soft tissue the device is applied to. Therefore, the cup can stay in place for seconds to weeks or months.

The cervical cup may or may not have a pressure sensor disposed therein or attached thereto in order to detect the actual pressure applied to the cervix tissue. A small, thin, flexible, wireless pressure sensor may be desirable. The cervical cup can be of any shape but should be tailored to the tissue that is aimed at being displaced/shaped. The cervical cups without a flange are also within the scope of the subject matter disclosed herein.

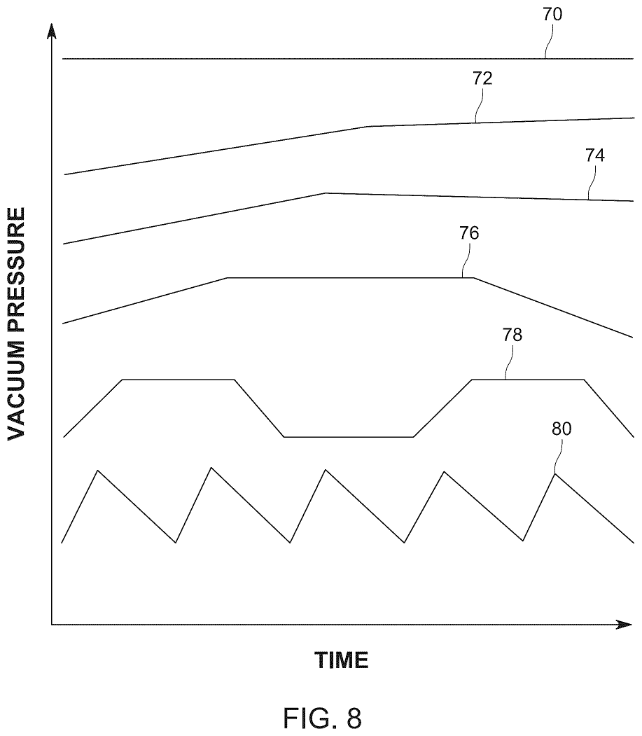

Those of ordinary skill in the applicable arts will appreciate that the system illustrated in FIG. 7 is capable of many different ways to apply negative pressure profiles to a cervix, including pressure profiles that will provide: (a) a static stretch, (b) a ramp stretch, (c) a ramp-and-hold stretch, (d) a ramp-hold-relaxation stretch, and (e) a cyclic stretch, to name just a few. All have the ability to increase the load-bearing qualities of the cervix. FIG. 8 illustrates some examples of generic pressure profiles.

The pressure profile 70 is for a static stretch applied to a cervix, wherein the holding of constant negative pressure on the cervix through the application of a simple cup through time, as described above. The benefits of a static stretch on collagen have been demonstrated in orthodontia, showing an increase in collagen synthesis and improvements in the load-bearing tissue that supports teeth.

A ramp stretch is illustrated in the pressure profile 72 of FIG. 8, wherein negative pressure increases (becomes more negative) with time. This can be achieved in several ways with the cervical cup applied to the cervix. This can be achieved by an incremental increase in cup size over time, by an increase in the amount of pressure over time with the same cup size, a combination of both, or a flexible, size-changing cup that increases with time. This can also be achieved by increasing the cup length, radius, or both over time.

The pressure profile 74 of FIG. 8 illustrates a ramp-and-hold stretch corresponding to increases of an applied negative pressure until a threshold amount is reached. Then, the pressure is held constant throughout the remainder of the pregnancy. This can be achieved by the above-mentioned profile used to achieve a ramp stretch and subsequently maintaining a desired measure of negative pressure for a particular length of time.

The pressure profile 76 corresponds to a technique of a ramp-hold-relaxation stretch that includes the steps of a ramp-and-hold stretch followed by a period of relaxation. This can also be achieved by an incremental increase in cup size over time (placing a small cup, removing the small cup, replacing it with a larger cup), by an increase in the amount of pressure over time with the same cup size, a combination of both, or a flexible, size-changing cup that increases with time. This can also be achieved by increasing the cup length over time, the cup radius over time, or both. This increase will be followed by a holding of the applied pressure amount, followed by a decrease in pressure with the same techniques—an incremental decrease in the cup size, a decrease in the amount of pressure over time, a combination of both, or a flexible, size-changing cup that decreases with time.

The pressure profile 78 of FIG. 8 corresponds to an embodiment where a cyclic stretch is applied to the cervix by a repeated pattern of stretch with intermittent periods of holding the applied pressure and a period of rest (no pressure applied). Such a pattern has many benefits for different collagen types. These benefits have been demonstrated in tendons, vessels, and other areas of the body that are frequently used in a cyclic manner. Benefits include the previously mentioned, such as increased collagen tensile strength, increased collagen synthesis, decreased collagen degradation, decreased adhesions, and decreased inflammatory mediators such as prostaglandins.

The pressure profile 80 of FIG. 8 corresponds to an embodiment where a ramp increase followed by a ramp decrease relaxation pattern of cyclic stretch is applied to the cervix. ed. As illustrated in FIG. 8, this is a pattern of immediate increase, followed by immediate decrease rather than including a period of hold, like in the pressure profile 78. This pressure profile also corresponds to an embodiment where a cyclic stretch is applied to the cervix by a repeated pattern of stretch that has many benefits for different collagen types and provides similar benefits as the ones listed above for the pressure profile 78 of FIG. 8. These benefits have been demonstrated in tendons, vessels, and other areas of the body that are frequently used in a cyclic manner.

It will be understood by those of ordinary skill in the relevant arts that the pumping control system 64 can be a portable device, thus allowing for the intermittent, cyclic stretch (as exemplified in the embodiments illustrated in FIG. 8) to be applied on a daily basis without the patient using the cervical cup being required to be hospitalized. The portable pumping control system is carried by the patient by strapping it to the patient's body, similar to a breast pump, in order to be portable during use.

Cyclic stretch with the cervical cup can be achieved in many ways: changes in cup size, changes in the amount of pressure with the same cup, a combination of both, or a size-changing cup that increases and decreases with time. These changes can be fast or slow—over seconds, minutes, days, weeks, or months. The intervals of change can be in a regular or irregular pattern. Any stretch pattern can be achieved with the cervical cup using a standard cup, a tilted cup, a curved cup, or a medicated ring cup. These many patterns of stretch can be used for part or all of the remaining pregnancy.

A pressure sensor embedded in the cup, comprising the whole cup, a part of the cup, or multiple spots within the cup can help detect the amount of pressure applied to the cervix by the cervical cup. A cervical cup made of a material that will change color based on the amount of pressure applied will allow one to visually detect changes in applied pressure.

Those of ordinary skill in the applicable arts will appreciate after review of the disclosed subject matter that the cervical cup, applied with gentle suction and then held in place by a check-valve system, is designed to create a moderate amount of negative pressure—tension—in order to stimulate the immediate stiffness of the cervix itself, followed by a long-term improvement in cervical function which includes increases collagen tensile strength, increases collagen synthesis, decreases in collagen degradation, decreases in adhesions, increases in collagen crosslinks, and decreases in prostaglandins. This improvement in the function of the cervix will decrease preterm birth among wearers.

Those of ordinary skill in the applicable arts will also appreciate after reviewing the subject matter disclosed herein that the embodiments disclosed can also provide pelvic floor support, create a barrier to infection, allow amniotic fluid to reaccumulate following early membrane rupture, and administer medications to further support the pregnancy.

The cervical cup also supports the pelvic floor and changes the uterocervical angle towards a beneficial position, creating cervical edema and restoring endocervical length. This is a paradigm shift for obstetrics as the cervical change was previously believed to be irreversible in nature, with researchers categorizing dilation as “ratchet-like.” This type of cervical change—changing the uterocervical angle towards a beneficial position, creation of cervical edema, and restoration of endocervical length—are all the types of change that the Cervical cup creates and sustains.

Toward the end of pregnancy and in the process of parturition, downward pressure on the internal os of the cervix created by the fetus, fetal membranes, amniotic fluid, and growing myometrium stimulates the body to release the hormone oxytocin, which stimulates contractions, creating greater pressure on the internal os of the cervix, continuing the positive feedback loop—called the Ferguson Reflex—that concludes with the expulsion of the uterine contents. Some of the early phases in this process can be disrupted by supporting the organs of the pelvis. The cervical cup offers this benefit along with the several other benefits mentioned earlier. The cervical cup also offers a mode of administering pregnancy-prolonging medications through a medicated ring that surrounds the cervix and administers medication via perforations in the cup flange. A healthcare provider may choose to medicate the patient for more comprehensive care simultaneously.

The cervical cup may protect against infection. An important factor in ensuring the success of a pregnancy is the maternal-infant dyad's ability to resist infection. Many adaptations during pregnancy assist the mother in protecting against infection. Those who develop infection, even sub-clinical, may consequently deliver preterm. One of the most important factors in preventing infection is the physical barrier created by the cervix, cervical mucous, and fetal membranes. This barrier is enhanced by the subject matter of this disclosure.

Preventing infection becomes even more important and more difficult in mothers who experience PPROM. Intrauterine infection is found in more than ⅓ of women who experience PPROM. If the dyad develops an infection associated with PPROM, the period between the rupture and the delivery time is much smaller. The neonatal mortality rate is also four times higher for those in whom infection is present following PPROM. This infection increases not only the risk and timing of preterm delivery but also the risks of neonatal sepsis, respiratory distress syndrome, chronic lung disease, periventricular leukomalacia (necrosis of brain tissue adjacent to the cerebral ventricles), intraventricular hemorrhage, and cerebral palsy in those infants that survive the infection. Because of these factors, it is desirable that the dyad's ability to fight infection be enhanced. The barrier that the cervical cup creates does just that.

Another issue that arises in cases of PPROM is the fetus'ability to continue to develop normally without adequate amniotic fluid. Fetal development without the presence of adequate amniotic fluid results in limb position defects and respiratory distress syndrome, secondary to pulmonary hypoplasia and prematurity. A physical barrier has been found to be beneficial to mothers experiencing PPROM by preventing the loss of amniotic fluid. The cervical cups disclosed herein provide this physical barrier that protects mothers and infants from pathogens and the dangers of low amniotic fluid while promoting cervical ripening resistance.

The cervical cups, according to the subject matter disclosed herein, can also administer medications, as already mentioned. Just as intravaginal rings have been used safely and efficiently to deliver drugs consistently over time, the cervical cup can be used in the same manner if needed. With the presence of perforations around the flange and a medicated ring insert, the cervical cup can disseminate medications throughout the length of its placement. These medications could be similar to tocolytics, oxytocin inhibitors, progesterone, or other hormones in the event that the medication is found beneficial to maintaining the pregnancy.

Various embodiments described may be used without a medicated ring to medicate the cervix or cyanoacrylate glue to hold the cervical cup in place. Some embodiments may focus on mothers with short cervices at 16-24 weeks of gestation. Other embodiments may be focused on mothers with a history of preterm birth at 13 weeks of gestation, mothers with a short cervix at 13-36 weeks of gestation, mothers who present with preterm labor symptoms, mothers who present with rupture of membranes, and may include a medicated ring and/or cyanoacrylate glue.

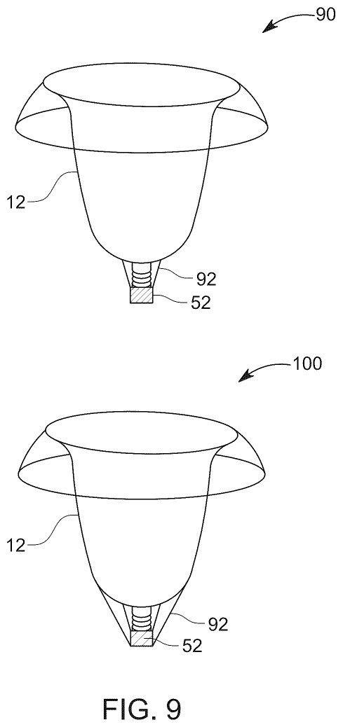

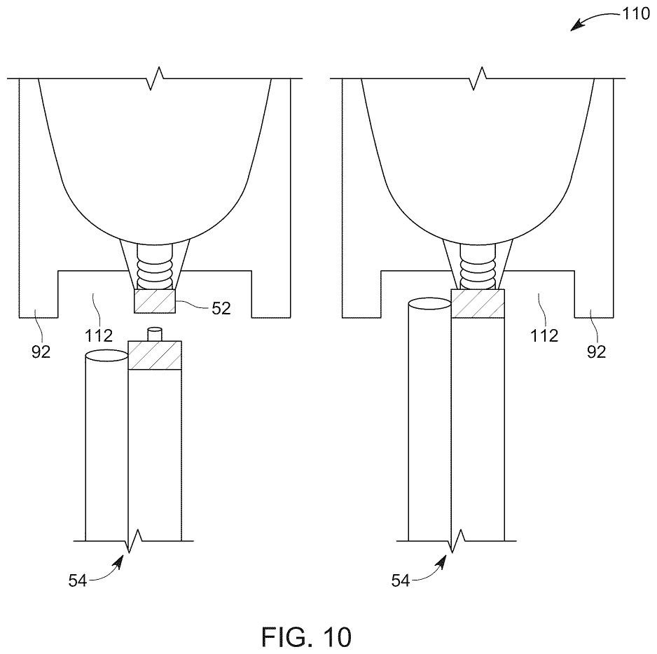

FIG. 9 illustrates embodiments of the cervical cups with streamlined tips to facilitate insertion in a patient. In the streamlined embodiment 90, a lip 92 extends from the bottom of the cervical cup 12 to a proximal edge of the valve 52. In the streamlined embodiment 100, lip 92 extends from the bottom of the cervical cup to a distal edge of valve 52 so as to envelop valve 52. FIG. 10 illustrates another streamlined embodiment 110 wherein a space 112 is provided between the lip 92 and the valve 52 so as to allow the vacuum line 54 to be inserted and rotated to connect with the valve 52, for example, for a luer-lock type of connection. In the embodiment illustrated in FIG. 10, a dual vacuum line 54 is illustrated, in which one of the lines is exposed to ambient air as a pressure reference as previously discussed.

Those of ordinary skill in the applicable arts will appreciate that a number of combinations of valve 52 and cervical cup 12 make it possible to close the vacuum inlet. Embodiments of valve 52 may include, but are not limited to, a normal check vacuum inlet with a check valve; no check valve; multiple inlets with one main check valve; multiple inlets with multiple check valves; a valve comprising a plunger solid cylinder that pushes an O-ring in place as the cervix tissue moves into place.

This system can also be integrated with biosensors that deliver information to smart devices, patients, healthcare providers, and/or data collection systems and electronic medical records systems. These biosensors can detect blood, amniotic fluid, temperature, notice of creep within the cup, and/or the incorporation of a camera.

Included in the subject matter disclosed herein is a flangeless cervical cup. One of the advantageous features of a flangeless cervical cup is the easiness of its insertion and removal. The absence of a flange makes the cup easier to place and remove because of its size, shape in relation to the cup portion, and its more compliant nature, which makes it more conducive to folding. From the patient's perspective, the flangeless cervical cup, with its reduced cross-sectional area, makes insertion and removal easier and less painful for users. If the cervical cups disclosed here are being used intermittently for cyclic stretch, the pelvic “lift” created by the flange is not needed, thus minimizing or even eliminating the need for a flange. If the inner diameter of the flange is smaller than the cervical diameter at the point at which they meet, the flange may create an unwanted positive pressure at the junction that may potentially break down the cervical tissue, leading to softening. For these reasons, having a cup without a flange may be beneficial. Therefore, a cervical cup is needed only once a day and for an hour to perform cyclic stretch (for example, every 3 minutes, the cervical cup pulls on the cervix and then releases and pulls again). In such situations, one wouldn't necessarily need a flange because you are not trying to create support for the pelvic floor but are just trying to give a “workout” to the cervix so it can get stronger.

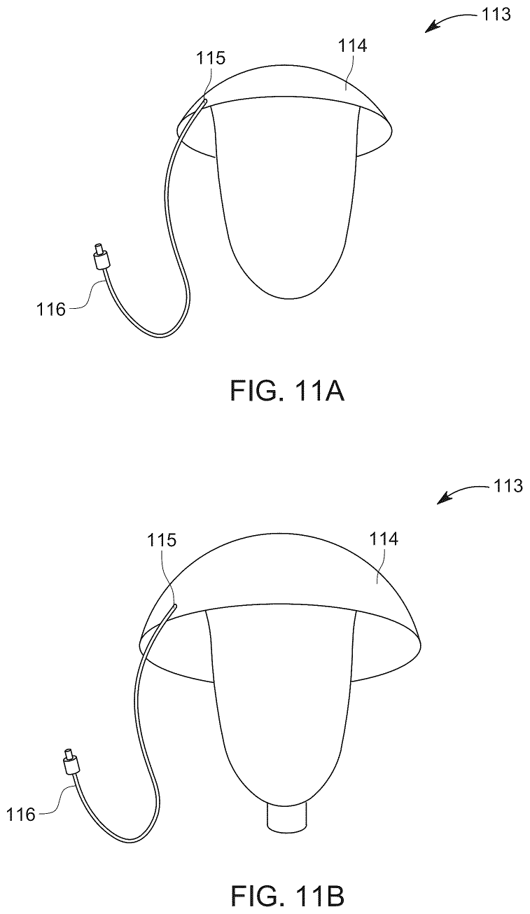

In yet another embodiment of the subject matter disclosed herein, the easiness of insertion and removal of cervical cups is accomplished by a cervical cup 113 that comprises an inflatable flange 114, as illustrated in FIG. 11. As shown, the cervical cup 113 includes the inflatable flange 114 with an inflation port 115 connected to an inflation hose 116 configured to supply air or liquid to inflate the flange 114. With the inflatable flange 114 deflated (as shown in FIG. 11 (A)), insertion and removal will be made easier and less uncomfortable for the patient. Once in place, the inflatable flange 114 can then be inflated with a gas or a liquid for proper functioning. In some embodiments, the inflation hose 116 may remain connected to the inflatable cervical cup 113 after inflation, while in other embodiments, the inflation hose 116 is removed once the flange is inflated.

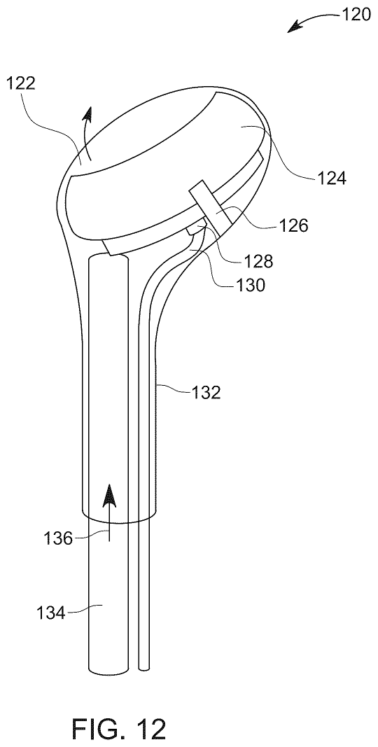

FIGS. 12-14 illustrate various embodiments of tools configured to insert the disclosed cervical cups within the vaginal vault of a patient with ease for cup placement. FIG. 12 shows an applicator 120 that comprises a slit 122 configured to accommodate a folded cervical cup 124 held in place by clips 126, the check valve 128 of the folded cup 124 being connected to the vacuum line 130. A handle 132 of the applicator is configured to accommodate the vacuum line 130 and a placement plunger 134. Once the folded cup is placed next to the cervix of the patient, the placement plunger 134 is pushed upward in the direction of the arrow 136, pushing the folded cup 124 from the clips 126 into the cervix, at what point the vacuum is applied to the vacuum line 130 so as to help the cup in place.

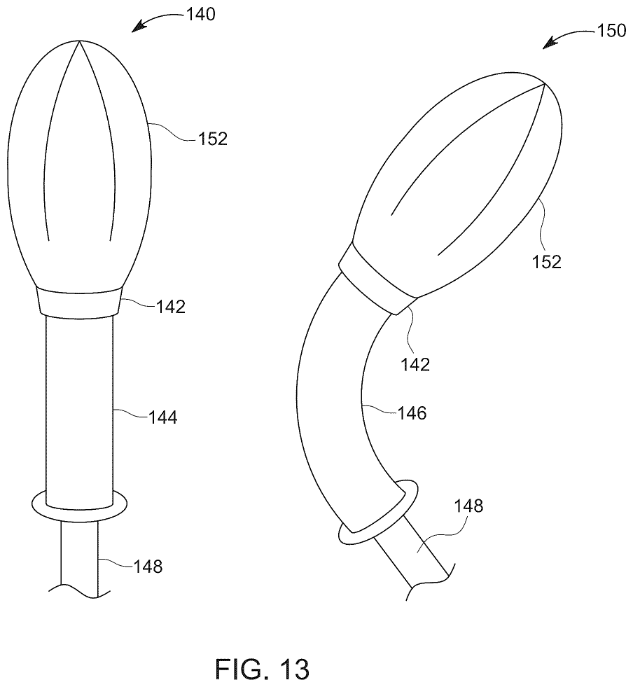

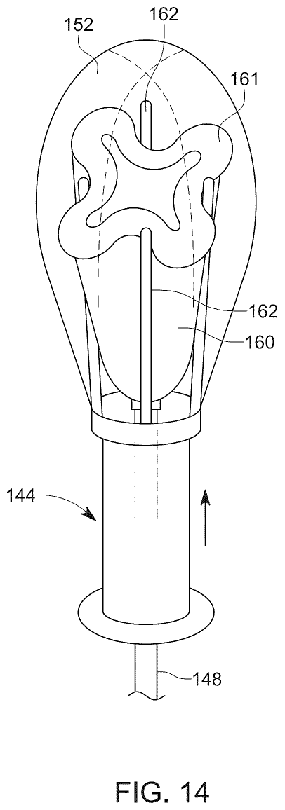

FIG. 13 illustrates straight (140) and curved (150) embodiments of another insertion tool. These applicators comprise an applicator ring 142, straight (144) and curved (146) plungers, and a vacuum line 148. Folded cervical cups are held inside of applicator petals 152 before insertion and placement. The structure of the petals 152 is better shown in FIG. 14, wherein a folded cervical cup 160 with a flange 161 is held in place by posts 162 inside of the applicator petal 152. In use, the tool 140 is inserted into the vaginal vault, and the plunger 144/146 is moved upward, releasing the cup 160 from the posts 162. Subsequent application of a vacuum to the suction line 148 will keep the cervical cup in place against the cervix of the patient receiving the pre-term prevention treatment.

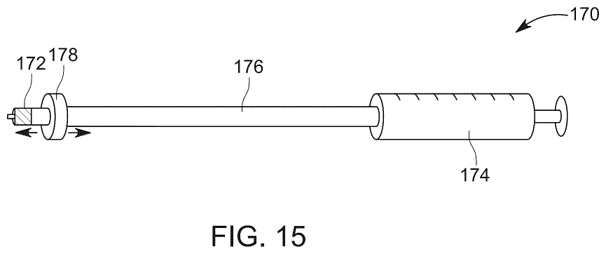

FIG. 15 illustrates an embodiment of a tool for removing a cervical cup held in place against the cervix of a patient by applying a vacuum. The tool 170 has a luer-type lock 172 to be connected to a corresponding luer-type lock in the cup (not shown). Once connected, a liquid or a gas is applied to the cup by a syringe 174 via a tube 176. Once the vacuum holding the cup in place is broken, a disc 178 disposed near the luer-type lock 172 is used the fold the cervical cup before removal via the vaginal vault of the patient. In order to provide more rigidity, the tube 176 may be comprise a guide wire (not shown) attached thereto, thereby facilitating the guidance of the tool 170 in and out of the vaginal vault.

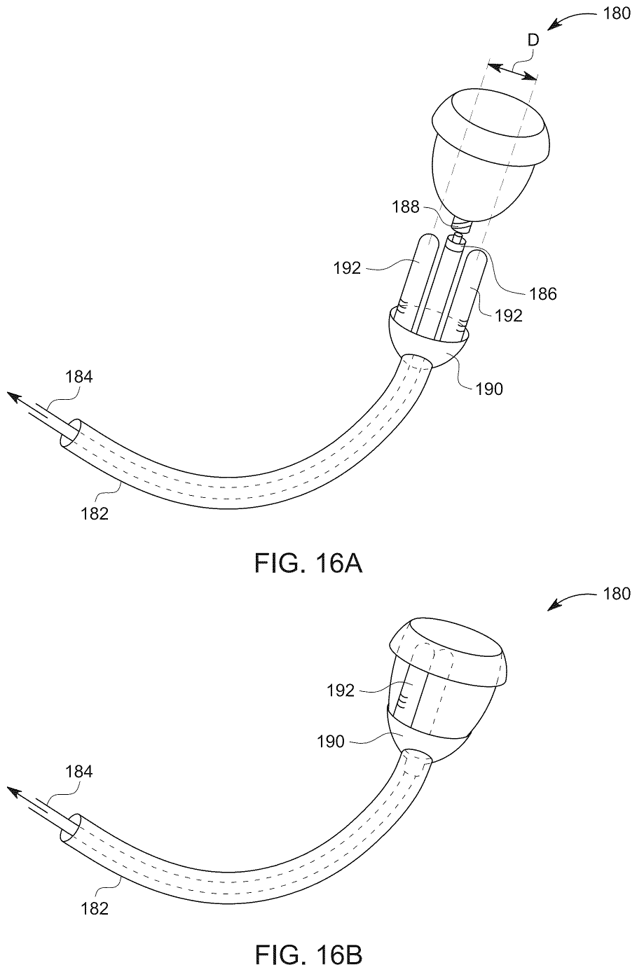

FIG. 16 illustrates another removal tool 180 according to the subject matter disclosed. This removal tool 180 comprises a curved handle 182 and a vacuum line 184 disposed inside of the curved handle 182. At the extremity close to the cervical cup to be removed, the vacuum line includes an attachment 186 configured to be connected to a valve 188 of the cervical cup so as to introduce a gas or a liquid inside of the cervical cup to break the vacuum. At the same extremity, the removal tool 180 includes a cup-like insert 190 that slides over the curved handle 182 of the tool. As shown, the cup-like insert 190 further includes two pins 192 separated from each other by a distance D which is smaller than the largest diameter of the cervical cup such that once the vacuum is released by the vacuum line 184 and the cup-like insert 190 is pushed against the cervical cup, the two pins 192 causes the cervical cup to change from a circular to an elliptical cross-sectional shape which will facilitate the removal of the cervical cup through the vaginal canal of the patient. The curvature of the handle 182 is designed to fit around the thigh and reach to the vagina. Once the cup valve 188 is manually screwed onto the vacuum line 184, the removal tool cup 190 and tubing 184 may be held in one hand (grooves may be placed to assist with easy handling), with the other hand pulling the tubing 184 and cup-like insert 190 up through the removal tool 180. The cervical cup will fold between the two pins 192 into the insert 190, which will facilitate the cup coming out easily out of the vagina at an angle, as well as allowing for the cup to fold in half which allows for the flange to collapse. This combination of the angle and cup folding is the least painful way found so far to remove the cup. A clamp can be put onto the end of the curve in order to hold the cervical in between the pins.

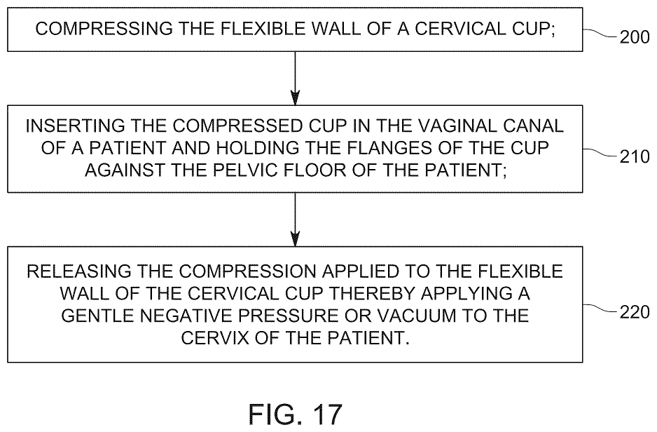

Methods and processes to treat preterm births using cervical cups as described are also within the scope of the subject matter disclosed. FIG. 17 illustrates the flowchart of an exemplary embodiment of such a method or process, which includes the following steps: (1) placing the mother in a lithotomy position; using sterile technique, slide the malleable cup into the vagina; place the cup on the cervix; employing the vacuum using a hand pump; capping off the vacuum apparatus, maintaining the seal of the vacuum; removing the vacuum apparatus; leaving the cup in place until term, pop off, or symptoms of labor; and cutting the suction tubing above the place of clamp or removing the clamp. When removing the cervical cup, the practitioner simply applies a sterile-gloved finger to the side of the cup to break the suction in order to remove the cup from the vagina.

The exemplary embodiments provide apparatuses, methods, and systems for treating preterm births, and it should be understood that this description does not limit the invention. On the contrary, the exemplary embodiments are intended to cover alternatives, modifications, and equivalents, which are included in the spirit and scope of the invention as defined by the appended claims. Further, in the detailed description of the exemplary embodiments, numerous specific details are set forth in order to provide a comprehensive understanding of the claimed invention. However, one skilled in the art would understand that various embodiments might be practiced without such specific details.

Although the features and elements of the present exemplary embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims and may include other examples that occur to those skilled in the art. Such other examples are intended to be within the scope of the claims.

While the disclosed embodiments of the subject matter described herein have been shown in the drawings and fully described above with particularity and detail in connection with several exemplary embodiments, it will be apparent to those of ordinary skill in the art that many modifications, changes, and omissions are possible without materially departing from the novel teachings, the principles and concepts set forth herein, and advantages of the subject matter recited in the appended claims. Hence, the proper scope of the disclosed innovations should be determined only by the broadest interpretation of the appended claims so as to encompass all such modifications, changes, and omissions. In addition, the order or sequence of any process or method steps may be varied or re-sequenced according to alternative embodiments. Finally, in the claims, any means-plus-function clause is intended to cover the structures described herein as performing the recited function and not only structural equivalents, but also equivalent structures.

What is claimed is:

1. A cervical cup, comprising:
a flexible body generally extending along a first direction along an axial axis thereof, said body being generally curved and open at a proximal end thereof; and
a flange attached to the proximal end portion of said flexible body;
wherein, said flange of the cervical cup is configured to be held against a pelvic floor of a patient being treated for preterm birth by a suction pressure, said suction pressure being generated by compressing the flexible body of the cervical cup before holding said flange against the pelvic floor and subsequently releasing the compressed flexible body.

2. The cervical cup, according to claim 1, further comprising:
circumferential fold lines disposed along the first direction of the flexible body, said circumferential fold lines being configured to allow the flexible body to be folded before applying the cervical cup to pelvic floor of the patient.

3. The cervical cup, according to claim 1, wherein said flexible body comprises an outer wall of the cervical cup, the cervical cup further comprising:
an inner wall having a plurality of suction holes;
a valve disposed at a distal end portion of the cervical cup, said valve being connected to both the outer and inner walls of the cervical cup so as to form an enclosure; and
a plurality of suction lines connecting the plurality of suction holes to the valve, wherein said suction pressure is generated by a vacuum source attached to the valve.

4. The cervical cup, according to claim 3, wherein a shape of a cervix disposed inside the cervical cup is altered by the location of the plurality of the suction holes in the inner wall.

5. The cervical cup, according to claim 4, wherein the plurality of suction holes is disposed near a distal portion of the inner wall so as to pull the cervix down in the first direction.

6. The cervical cup, according to claim 4, wherein the plurality of suction holes is disposed near a proximal portion of the inner wall so as pull the cervix sideways in a second direction along a radial axis of the flexible body.

7. The cervical cup, according to claim 3, further comprising:
a vacuum control system having a vacuum pump and a suction line connecting the vacuum pump to the valve of the cervical cup, wherein the vacuum control system is configured to generate a time dependent suction profile.

8. The cervical cup, according to claim 7, wherein said time dependent suction profile is selected from a group comprising a static stretch, a ramp stretch, a ramp-and-hold stretch, a ramp-hold-relaxation stretch, or a cyclic stretch.

9. The cervical cup, according to claim 7, wherein a position of the cervix inside of the cervical cup is determined by a pressure differential between a first suction hole and a second suction hole, the first suction hole being connected to the valve and the second suction hole being connected to a reference pressure source.

10. The cervical cup, according to claim 9, wherein the reference pressure source is an ambient pressure.

11. The cervical cup, according to claim 1, wherein said flexible body comprises an outer wall of the cervical cup, the cervical cup further comprising:
an inner wall having first and second suction holes;
a valve disposed at a distal end portion of the cervical cup, said valve being connected to both the outer and inner walls of the cervical cup so as to form an enclosure, said valve also being connected to the first and second suction holes; and
first and second suction lines connecting the first and second suction holes through the valve to first and second pressure sources, wherein said suction pressure is generated by the first and second pressure sources.

12. The cervical cup, according to claim 11, wherein a position of the cervix inside of the cervical cup is determined by a pressure differential between the first and second suction lines.

13. A method to treat preterm birth, the method comprising:
compressing a flexible body of a cervical cup, said flexible body generally extending along a first direction along an axial axis thereof, said body being generally curved and open at a proximal end thereof;
placing a flange attached to the proximal end portion of said flexible body against a pelvic floor of a patient; and
generating a vacuum between the cervical cup and a cervix of the patient by releasing the compressing of the flexible body.

14. The method, according to claim 13, further comprising:
folding the flexible body along circumferential fold lines disposed on the flexible body before said compressing.

15. The method, according to claim 13, further comprising:
generating said vacuum by connecting a vacuum source to a valve of the cervical cup, said valve being disposed at a distal end portion of the cervical cup, wherein said flexible body forms an outer wall of the cervical cup, the cervical cup further comprises an inner wall with a plurality of suction holes, and a plurality of suction lines connecting the plurality of suction holes to the valve; and
closing the valve.

16. The method, according to claim 15, further comprising:
applying a time dependent suction profile to the valve with a vacuum control system having a vacuum pump and a suction line connecting the vacuum pump to the valve of the cervical cup, wherein the vacuum control system is configured to generate a time dependent suction profile selected from a group comprising a static stretch, a ramp stretch, a ramp-and-hold stretch, a ramp-hold-relaxation stretch, or a cyclic stretch.

17. The method, according to claim 15, further comprising:
generating said vacuum by connecting a vacuum source to a valve of the cervical cup, said valve being disposed at a distal end portion of the cervical cup, wherein said flexible body forms an outer wall of the cervical cup, the cervical cup further comprises an inner wall with first and second suction holes, and first and second of suction lines connecting the first and second suction holes to first and second pressure sources; and
closing the valve.

18. The method, according to claim 17, further comprising:
determining a position of the cervix inside of the cervical cup by a pressure differential between the first and second suction lines.

19. The method, according to claim 15, wherein the plurality of suction holes is disposed near a distal portion of the inner wall, the method further comprising:
shaping the cervix by pulling the cervix down in the first direction by the applied vacuum pressure.

20. The method, according to claim 15, wherein the plurality of suction holes is disposed near a proximal portion of the inner wall, the method further comprising:
pulling the cervix sideways along a radial direction of the flexible body by the applied vacuum pressure.
